(19) United States
(12) Patent Application Publication
(10) Pub. No.: US 2005/0220851 A1
(43) Pub. Date: Oct. 6, 2005

(54) Matrix type patch containing bronchodilators

(76) Inventors: Young Kweon Choi, Seoul (KR); Hyun Suk Yu, Kunpo-shi (KR); Il Gyun Shin, Seoul (KR); Hee Sook Kim, Seoul (KR)

Correspondence Address: LADAS & PARRY, 26 WEST 61ST STREET, NEW YORK, NY, 10023, US

(21) Appl. No.: 10/513,239

(22) PCT Filed: May 16, 2003
(86) PCT No.: PCT/KR03/00975

§ 371 Date: Apr. 26, 2005

(30) Foreign Application Priority Data
May 20, 2002 (KR) 10-2002-0027818

(51) Int. Cl. A61K 9/70

(52) U.S. Cl. 424/449

(57) ABSTRACT

The present invention relates to a matrix type patch for the transdermal delivery of a bronchodilator which is consisted of a backing layer, a pressure-sensitive adhesive layer comprising a drug, and a release liner, wherein the said pressure-sensitive adhesive layer comprises as the drug component at least one of brochodilators selected from a group consisting of formoterol, salmeterol, tulobuterol, clenbuterol and the like; cationic polymers enhancing skin-permeability of the said drug components; solubilizing agents of the drug components; and pressure sensitive adhesives. The matrix type patch according to the present invention provides high skin permeability of the drug by using cationic polymeric absorption enhancers, sustained duration of the drug action for a prolonged time and excellent adhesion to the skin without causing irritation.

### TECHNICAL FIELD

The present invention relates to a matrix type patch for treating respiratory diseases such as asthma, which provides high skin permeability of the drug by using cationic polymeric absorption enhancers. It also exerts sustained release of a drug, maintains an excellent adhesion to the skin.

### BACKGROUND ART

Asthma is a chronic respiratory disease accompanied by various symptoms such as shortness of breath, cough, wheeze, sputum, chest tightness and the like.

Recently asthma prevalence is increasing especially in children and there is a general trend of increased deaths and hospitalizations from asthma. The high mortality is often caused by bad compliance of the pharmacotherapy.

Guidelines for treating asthma is to use long-term control medications to maintain control of persistent asthma, and quick relief medications to treat symptoms and exacerbations.

Drugs to be used in treatment of asthma generally include bronchodilators such as beta-2 adrenergic agonists (β₂-agonists) which have selective actions on β₂-receptors in bronchial smooth muscle. Among them, formoterol has a higher selectivity on β₂-adrenergic receptor, faster onset of action, cost effectiveness, and relatively lower side effect to heart than other β₂-agonists.

However, most antiasthma drugs are commercialized in the types of oral medication, intravenous medication and inhalation. Since oral medication has some disadvantages i.e. that drugs are easily decomposed in liver by first-pass hepatic metabolism after internally delivered, and that there is an excess high blood concentration of drugs temporally after administration, and that it can cause gastrointestinal disturbance. In addition, oral antiasthma drugs are to be administered 2-3 times a day to keep pharmacological effect for the control of asthma symptoms.

Long-acting β₂-agonists are mostly available as oral dosage forms or inhalers, and the administration of which can be problematic especially in children and elderly patients. Considering the inconvenience of oral and inhaling administration to children and elderly patients, long-term treatment needed to control persistent asthma, side effects and possible tolerance due to a sudden increase of blood level after administration, a new asthma therapeutic preparation making a long-term treatment easy and convenient by reducing dose frequency as well as maintaining an effective blood concentration for a predetermined period, is required. A transdermal patch is suitable for this purpose and it is also advantageous to prevent the occurrence of the nocturnal asthma attack and exercise-induced asthma.

As a prior art regarding a percutaneous preparation containing antiasthma drugs, PCT WO 97/14411 discloses that the micro-crystalline-state drugs in the adhesive can provide a longer duration of drug action by loading the drug in excessive amount above the solubility. But, the duration of drug action of the said preparation is unsatisfactorily short as 24 hours. In addition, adhesive strength and permeability of a drug are undesirably reduced in the course of time owing to the crystallization of the drug which is readily formed in the adhesive layer.

Korean Patent Application Laid-open No. 1999-0062986 discloses a preparation containing a selective use of acrylic or rubber-based adhesive having high solubility to the drug or a use of a large amount of solubilizing agents such as isopropyl myristate, in order to dissolve at least 5% by weight of tulobuterol in the adhesive completely. However, the duration of drug action in the said preparation is limited to 24 hours. For the said preparation contains a large amount of solubilizing agents, cohesion of the adhesive layer deteriorates and the residue upon removing the preparation from the skin is remained.

Generally, since the bronchodilators as disclosed in the said prior arts have low solubility to the conventional adhesives, it is difficult to dissolve the drugs homogeneously in adhesives. And, although the drug may be homogeneously dissolved, the desired patch is hard to be designed, because the said bronchodilators especially in the salt form have very low transdermal permeability, which renders the drug efficacy delayed.

Accordingly, the transdermal permeability of the bronchodilators should be elevated by using adequate absorption enhancer in order to deliver drugs effectively through skin. As a prior art regarding this percutaneous preparation, Korean Patent Application Laid-open No. 1998-015057 discloses a transdermal patch comprising clenbuterol, solubilizing agents and absorption enhancers selected from a group consisting of diethyl toluamide, dimethyl formamide, N-methyl-2-pyrrolidone, propylene glycol and the like; and a method for preparing the patch. Absorption enhancers and low molecular polar solvents used in the said preparation may induce skin irritation, and some compounds are undesirably vaporized in the course of preparation owing to their low volatile temperature.

U.S. Pat. No. 5,079,008 discloses a matrix-type monolithic system comprising an impermeable backing layer; an adhesive layer containing formoterol produced by using 1,8-cineol and N-methyl-2-pyrrolidone as absorption enhancers; and a release liner. Further, in order to solve the disadvantages of the instability and low permeability of the drugs which are encountered in U.S. Pat. No. 5,079,008, U.S. Pat. No. 6,211,425 discloses a formoterol patch using N-methyl-2-pyrrolidone as a solvent of ethylene/vinylacetate copolymer, and isopropyl myristate or L-menthol as a plasticizer and/or a filler for enhancing absorption of the drug.

There is another prior art to improve the skin permeability of the drugs in salt forms by adding physiologically stable acidic or basic materials to transform the drugs to free base form. The suitable acidic or basic materials include compounds such as citric acid, succinic acid, tartaric acid, maleic acid, fumaric acid, benzoic acid, salicylic acid and lactic acid; inorganic base such as sodium hydroxide and potassium hydroxide; amines such as triethylamine, triethanolamine, diethanolamine and triisopropanolamine; or basic amino acids such as arginine.

U.S. Pat. No. 5,834,010 relates to a percutaneous preparation produced by adding triacetine as an absorption enhancer to basic drugs having at least 8.0 of dissociation constant (pKa) and then blending them with pressure-sensitive adhesive. U.S. Pat. No. 6,255,502 describes a percutaneoous, mucous absorption preparation containing basic drugs or their salts with bile acid or fatty acid.

However, the said prior arts are providing the use of low molecular weight acidic materials or strong organic solvents or organic amines as solublizing agents to increase the solubility of the drugs in adhesive layer and skin permeability. But, when they are used in a large amount, these materials not only induce skin irritation but also deteriorate structural properties such as cohesion and adhesion due to the reduced amount of adhesives. Accordingly, it is difficult with the said solublizing agents to produce percutaneous preparation which can sustain an effective blood level of drugs for a prolonged period of time, since they hardly maintain the adequate structural property as percutaneous preparation and show sudden decrease of adhesive strength due to the reduced amount of adhesives.

### DISCLOSURE OF THE INVENTION

Therefore, the object of the present invention is to provide a matrix type patch which overcomes the delayed release of the drug, low permeability through the skin, and low adhesion of the adhesive layer to the skin.

In the present invention, cationic polymers with alkylamino group have been found to have high transdermal absorption enhancing effect for formoterol, tulobuterol, clenbuterol and the like, which was comparable to the conventional low molecular weight percutaneous absorption enhancers. The matrix patch formulation of the present invention has been obtained on the basis of this finding, and the production is improved in the drug solubility to the adhesive mixture and the permeation through the skin by using the proper solubilizing agents and permeation enhancers, which is adequate for the patch to exert pharmacological effect for a long time. It also has a good long-term stability of the drug, excellent cohesive and adhesive strengths without causing skin irritation.

### BRIEF DESCRIPTION OF THE DRAWINGS

The above object, other features and advantages of the present invention will become more apparent by describing the preferred embodiment thereof with reference to the accompanying drawings, in which:

FIG. 1 is a schematic drawing illustrating a patch according to the present invention;

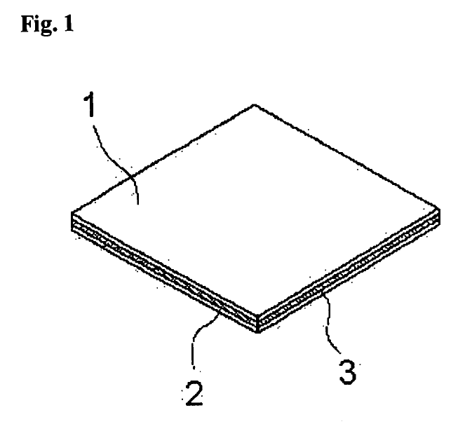

FIG. 2 is a graph showing time course of the drug permeation through human cadaver skin from the patch.

### BEST MODE FOR CARRYING OUT THE INVENTION

A matrix type patch according to the present invention comprises an impermeable backing layer (1), a pressure-sensitive adhesive layer containing drugs (2) and a release liner (3), wherein the said pressure-sensitive adhesive layer comprises as the drug component at least one of bronchodilators selected from a group consisting of formoterol, salmeterol, tulobuterol, clenbuterol and the like; a cationic polymer enhancing skin-permeation of the said drug component; solubilizing agents of the drug component; and pressure sensitive adhesives. The drug to be used in the present invention may be not only bronchodilators themselves but also salts thereof.

The backing layer to be used in the present invention should be impermeable to the drugs and the pharmaceutical excipients such as solubilizing agent, absorption enhancer and the like, should be thin and flexible, and have no reaction with adhesive layer. There are generally-used films made of polyester, polyethylene, polypropylene, polyolefin, polyethylene terephthalate, polyurethane, aluminium-deposited polyester and the like.

Moreover, the backing layer may be subjected to corona discharge treatment, plasma treatment, oxidation treatment and the like to increase adhesion with the said adhesive layer. Considering the requirement that the patch should be adhered to the skin for a prolonged period of time so as to exert the long-lasting drug effect, it is more preferable to laminate non-woven fibers, cotton fibers, woven fibers and the like having moisture absorbability onto the said films to prevent the patch from detachment owing to the moisture vaporized from the skin.

In the matrix type patch according to the present invention, bronchodilators as the drug component should be limited to formoterol (daily oral dose: 160 μg), salmeterol (daily inhalation dose: 100˜200 μg), tulobuterol (daily oral dose: 2,000 μg,), and clenbuterol (daily oral dose: 40 μg), since the said components have high selectivity on β₂-adrenergic receptor regarding the β₂-agonists representing bronchial relaxation and thus have excellent antiasthmatic effect.

The content of the drug component to be used in the present invention is 0.1˜10% by weight, preferably 1˜5% by weight on the basis of the total weight of the adhesive layer. If the content is less than 0.1% by weight, the effect of the drug is not sufficient since the skin-permeation rate of the drug is reduced. In the meantime, if the content is more than 10% by weight, it is difficult to design the patch since the drug is crystallized or deteriorates cohesion and adhesion of the adhesive layer.

In the matrix type patch according to the present invention, the polymeric base materials to be used in the adhesive layer (2) include acrylate polymers; rubber-based polymers such as polyisobutylene, polyisoprene, styrene-isoprene-butadiene copolymer, styrene-butadiene copolymer and the like; and polydimethylsiloxane based silicone polymers which are generally used as a pressure-sensitive medical adhesive.

The content of the adhesives to be used as the base materials in the present invention is 40˜90% by weight on the basis of the total weight of the adhesive layer. If the content is less than 40% by weight, mechanical properties or adhesion adequate as adhesive base material cannot be obtained. Meanwhile, if the content is more than 90% by weight, the skin-permeation is lowered and the duration of the drug action is short, since the content of polymeric absorption enhancers and solubilizing agents are proportionally lowered.

In the matrix type patch of the present invention, cationic polymeric absorption enhancers to be used in order to increase transdermal permeability of the drugs include homopolymers or copolymers, for example, (meth)acrylic monomers having mono-, di-, tri-alkylamino group such as dimethylaminoethyl acrylate (DMAEA), dimethylaminoethyl methacrylate (DMAEMA) and dimethylaminopropyl acrylamide (DMAPAAm); styrene monomers having mono-, di-, tri-alkylamino group such as dimethylamino styrene (DMASt) and dimethylaminoethyl styrene (DMAESt).

It is preferable to use one or two cationic polymers selected from poly(vinylpyrrolidone-co-dimethylaminoethyl methacrylate) (COPOLYMER 958, ISP TECHNOLOGIES, INC., U.S.A.), poly(vinylpyrrolidone-co-vinylcaprolactam-co-dimethylaminoethyl methacrylate) (GAFFIX VC-713, ISP TECHNOLOGIES, INC., U.S.A.) and poly(vinylpyrrolidone-co-methacrylamidopropyl trimethyl ammoniumchloride) (GAFQUAT HS-100, ISP TECHNOLOGIES, INC., U.S.A.).

The content of the cationic polymeric absorption enhancers to be used in the present invention is 1˜20% by weight on the basis of the total weight of the adhesive layer. If the content is less than 1% by weight, skin permeation enhancing effect is not sufficient. Meanwhile, if the content is more than 20% by weight, adequate adhesiveness and skin permeability of the drug can be hardly controlled, since the content of drug component, adhesive base materials and solvents are proportionally lowered.

These cationic polymers have to be understood as including their salts, and may be used with bronchodilators as well as other therapeutic agents.

The solubilizing agents used in the matrix type patch of the present invention may be one or more solvents selected from N-methyl-2-pyrrolidone, laurylpyrrolidone, triethanolamine, propylene glycol, glycerine, triacetine, diethyleneglycol monoethylether, the conventional fatty acids or fatty alcohol derivatives. These solubilizing agents increase the solubility of the drug in adhesive. Accordingly, the solubilizing agents prevent the crystallization of the drug in adhesive to bring an increase of effective drug concentration in adhesive, thereby to increase the skin permeability of the drug.

The content of the said solubilizing agents is 1˜25% by weight on the basis of the total weight of the adhesive layer. If the content is less than 1% by weight, the drug may be crystallized. Meanwhile, if the content is more than 25% by weight, physical properties such as adhesion of the adhesive, layer are not adequate as a patch and the residue is remained in the skin after at removing the patch from the skin.

In the matrix type patch according to the present invention, release liner (3) is made of a thin polyethylene or polyester film coated with silicone or fluorine compounds. The said release liner also prevents release of the drug contained in adhesive layer during storage with its being attached to adhesive layer (2), and is removed prior to use.

The materials used in release liner (3) may be any ones or any types which are conventionally used in the percutaneous preparation.

Also, in the matrix type patch according to the present invention, other materials may be selectively used, for example, polymeric additives which are conventionally used in the field of the present invention to increase cohesive and adhesive strengths such as polyvinyl alcohol, hydroxypropyl cellulose, carboxylmethyl cellulose, polyacrylate, polyvinyl pyrrolidone, poly(vinyl pyrrolidone-co-ethylenevinyl acetate) (PVP/VA I-735, I-535, I-335, ISP, U.S.A.) and the like; adhesiveness-providing agents such as rosin-based resin, polyterpene-based resin, petroleum-based resin, terpenphenol-based resin and the like; plasticizers such as liquid polybutene, liquid polyacrylate, mineral oil, lanolin and the like; and fillers, for example, inorganic materials such as bentonite, talc, zinc oxide, calcium oxide, silica gel, kaolin, titanium oxide and the like.

In addition to these materials, conventional fatty acid, fatty acid alcohol, fatty acid ester, pyrrolidone derivatives and the like may be optionally used in order to increase skin permeability of the drug. Further, aromatics, preservatives, antioxidants, stabilizers, pigments and the like may be added thereto within the pharmaceutically allowable range.

The present invention is described in more detail by Examples and Comparative Examples, but the Examples are only illustrative and, therefore, not intended to limit the scope of the present invention.

### EXAMPLE 1

The matrix type patch of the present invention was prepared as follows.

400 mg of formoterol fumarate as a drug was added to 4 ml of methanol and completely dissolved to give a homogeneous solution. 1.4 g of vinylpyrrolidone-dimethylaminoethylmethacrylate copolymer {poly (vinylpyrrolidone-co-dimethylaminoethylmethacrylate): “COPOLYMER 958”, manufactured by ISP TECHNOLOGIES, INC., U.S.A.} as cationic polymeric absorption enhancer; 2 g of laurylpyrrolidone and 0.4 g of triethanolaamine as solubilizing agents; 0.6 g of vinylpyrrolidone-ethylenevinyl acetate copolymer {poly(vinylpyrolidone-co-ethylenevinylacetate), PVP/VA I-735, I-535, I-335, manufactured by ISP TECHNOLOGIES, INC., U.S.A.} as polymeric additives to increase adhesion and cohesion of the adhesive layer; and 15.1 g of acrylic adhesive polymer solution “Duro-TAK 87-4098” (manufactured by National Starch Co., U.S.A.) as base material which is used with 50 mg of butylated hydroxytoluene (BHT) and 50 mg of butylated hydroxyanisole (BHA) as antioxidants were added to the said mixture, stirred sufficiently for 1 hour to obtain an adhesive solution containing drug.

The obtained adhesive solution was applied on a polyester release liner by using a knife doctor, dried by gradually raising a temperature from 60° C. to 100° C. Aluminium-deposited polyethylene film (Schotchpak 1109, manufactured by 3M) was covered on the said film, and then cut into a size of 20 cm² to obtain a final product.

### EXAMPLES 2˜9

The compositions(%) of ingredients used in Examples 2˜9 are in Table 1 and the procedures were same as defined in Example 1.

### COMPARATIVE EXAMPLES 1 to 3

Transdermal Patches were produced by conducting the same way as in Examples 1˜9 of the present invention and the compositions(%) of Comparative Examples 1 to 3 are in Table 1.

TABLE 1

| | | | | | | | | | | Com. | | |
|---|---|---|---|---|---|---|---|---|---|---|---|---|
| | Examples | | | | | | | | | Examples | | |
| | 1 | 2 | 3 | 4 | 5 | 6 | 7 | 8 | 9 | 1 | 2 | 3 |
| drugs(%) | | | | | | | | | | | | |
| formoterol | 2 | 2 | 2 | | | | 2 | 2 | 2 | 2 | 2 | 5 |
| fumarate | | | | | | | | | | | | |
| salmeterol | | | | 2 | | | | | | | | |
| clenbuterol | | | | | 1 | | | | | | | |
| tulobuterol | | | | | | 4 | | | | | | |
| polymeric | | | | | | | | | | | | |
| absorption | | | | | | | | | | | | |
| enhancers(%) | | | | | | | | | | | | |
| Copolymer 958* | 7 | | | 7 | 5 | 10 | | 7 | 5 | | | |
| Gaffix VC-713* | | 7 | | | | | 10 | | | | | |
| Gafquat HS-100* | | | 7 | | | | | | | | | |
| solubilizing | | | | | | | | | | | | |
| agents(%) | | | | | | | | | | | | |
| laurylpyrrolidone | 10 | | 15 | 10 | 15 | 10 | 5 | 5 | 10 | 10 | | |
| triethanolamine | 2 | 2 | | 2 | | 5 | | 5 | 2 | 2 | | |
| N-methyl-2- | | 10 | | | | | | | | | 10 | |
| pyrrolidone | | | | | | | | | | | | |
| other additives(%) | | | | | | | | | | | | |
| PVP/VA I-735* | 3 | 5 | 7 | 3 | | 5 | | 5 | | 3 | | |
| rosin ester resin | | | | | | | | | 10 | | | |
| anitioxidants | 0.5 | 0.5 | 0.5 | 0.5 | 0.5 | 0.5 | 0.5 | 0.5 | 0.5 | 0.5 | 0.5 | 0.5 |
| (BHT/BHA) | | | | | | | | | | | | |
| low molecular | | | | | | | | | | | | |
| absorption | | | | | | | | | | | | |
| enhancers(%) | | | | | | | | | | | | |
| isopropyl | | | | | | | | | | | | 40 |
| myristate | | | | | | | | | | | | |
| 1-menthol | | | | | | | | | | | 20 | |
| adhesive base | | | | | | | | | | | | |
| materials(%) | | | | | | | | | | | | |
| acrylic adhesive | 75.5 | 73.5 | 68.5 | 75.5 | 88.5 | 65.5 | 82.5 | | | 82.5 | 67.5 | |
| 1* | | | | | | | | | | | | |
| acrylic adhesive | | | | | | | | 75.5 | | | | |
| 2* | | | | | | | | | | | | |
| rubber-based | | | | | | | | | 75.5 | | | 54.5 |
| adhesive* | | | | | | | | | | | | |

*Copolymer 958: poly(vinylpyrrolidone-co-dimethylaminoethyl methacrylate) (ISP, U.S.A.)
*Gaffix VC-713: poly(vinylpyrrolidone-co-vinylcaprolactam-co-dimethylaminoethyl methacrylate) (ISP, U.S.A.)
*Gafquat HS-100: poly(vinylpyrrolidone-co-methacrylamidopropyl trimethyl ammonium chloride) (ISP, U.S.A.),
*PVP/VA I-735: poly(vinylpyrrolidone-co-ethylenevinylacetate) (ISP, U.S.A.)
*acrylic adhesive 1: Duro-TAK 87-4098 (National Starch Chem. Co., U.S.A.)
*acrylic adhesive 2: Gelva 737 (Solutia, Monsanto, U.S.A.)
*rubber-based adhesive: Duro-TAK 87-3500 (National Starch Chem. Co., U.S.A.)

Experiment 1: Skin Permeation Experiment

The skin permeability of the drugs was determined to evaluate the efficacy of the patches of Examples 1 to 9 and Comparative Examples 1 to 3.

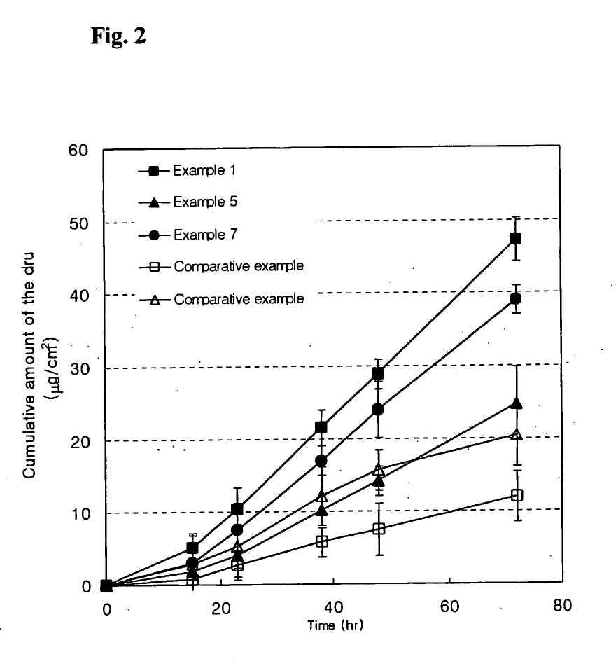

Receptor phase of Franz diffusion cell was filled with phosphate-buffered solution (pH 7.4) and maintained at temperature of 32±0.5° C. The patches of Examples and Comparative Examples were suitably cut into a size of d diffusion cell, and then attached to human cadaver skin. At the predetermined time intervals each 300 μl of the receptor solution was taken out to measure the amount of the drug permeated through the skin by a liquid chromatography. The results are shown in Table 2 and FIG. 2.

The skin permeation rates of the patches of Examples were about 24 times higher than those of the patches of the present invention of Comparative Example 1 which has no cationic polymeric absorption enhancer.

The patch of produced in Comparative Example 3 showed relatively higher skin permeation rate at an initial time of attachment, but showed gradually decreased skin permeation rate after 48 hours. Further, it undesirably induced skin irritation due to a large amount of fatty acid derivatives contained therein.

TABLE 2
Results of skin permeation through human cadaver skin

| | skin permeation | |
|---|---|---|
| | (15˜72 hrs) (μg/cm²/hr) | Enhancement ratio* |
| Example 1 | 0.754 | 4.0 |
| Example 2 | 0.546 | 2.9 |
| Example 3 | 0.675 | 3.6 |
| Example 4 | 0.642 | 3.4 |
| Example 5 | 0.424 | 2.2 |
| Example 6 | 1.177 | 6.2 |
| Example 7 | 0.645 | 3.4 |
| Example 8 | 0.677 | 3.6 |
| Example 9 | 0.523 | 2.8 |
| Com. Example 1 | 0.189 | 1.0 |
| Com. Example 2 | 0.319 | 1.7 |
| Com. Example 3 | 0.304 | 1.6 |

note)
*“Enhancement ratio” means relative skin flux of examples to that of Comparative Example 1.

Experiment 2: Skin Irritation Test

Skin irritation of the area treated with the patches of Examples and Comparative Examples was evaluated. The patches of Examples and Comparative Examples were cut into a size of 2.5 cm² and then attached to the upper parts of the chests of the healthy adult subjects for 24 hours. Skin response was evaluated after 24 hours and 72 hours. The intensity of irritation was determined according to the method of “Primary Irritation Index (P.I.I.)” of Draize method. The results are shown in Table 3.

Evaluation of Skin Irritation According to P.I.I.

| | |
|---|---|
| 0: | no irritation |
| less than 2: | low irritation |
| 2˜5: | medium irritation |
| more than 5: | serious irritation |

TABLE 3
Results of Skin Irritation Test

| | number of | Skin irritation index |
|---|---|---|
| | subjects | (P.I.I.) |
| Example 1 | 6 | 0.43 |
| Example 2 | 6 | 0.25 |
| Example 3 | 6 | 0.34 |
| Example 4 | 6 | 0.61 |
| Example 5 | 6 | 0.63 |
| Example 6 | 6 | 0.93 |
| Example 7 | 6 | 0.38 |
| Example 8 | 6 | 0.82 |
| Example 9 | 6 | 0.36 |
| Com. Example 1 | 6 | 0.32 |
| Com. Example 2 | 6 | 1.79 |
| Com. Example 3 | 6 | 2.38 |

As shown in Table 3, the matrix type patches of the present invention showed lower skin irritation and more excellent safety than those of Comparative Examples 2 and 3.

### INDUSTRIAL APPLICABILITY

The present invention provides a patch for treating respiratory disease such as asthma with a high skin permeation of bronchodilators by using cationic polymeric absorption enhancers.

The antiasthmatic drug action of the patch is efficacious for a prolonged time without deterioration of its adhesive or cohesive str engths and it hardly has skin irritation.

1. A matrix type patch for treating respiratory diseases such as asthma comprising an impermeable backing layer, an adhesive layer and a release liner, wherein the said adhesive layer comprises
(a) at least one of bronchodilators selected from a group consisting of formoterol, salmeterol, tulobuterol and clenbuterol;
(b) cationic polymers as absorption enhancer of the said bronchodilators;
(c) solubilizing agents for the said bronchodilators; and
(d) pressure sensitive adhesives.

2. A matrix type patch for treating respiratory diseases such as asthma according to claim 1, wherein the said cationic polymer is at least one homopolymers or copolymers selected from a group consisting of (meth)acrylic monomers having mono-, di-, tri-alkylamino group; styrene monomers having mono-, di-, tri-alkylamino group.

3. A matrix type patch for treating respiratory diseases such as asthma according to claim 2, wherein the said (meth)acrylic monomer having mono-, di-, tri-alkylamino group is dimethylaminoethyl acrylate, dimethylaminoethyl methacrylate or dimethylaminopropyl acrylamide; the said styrene monomer having mono-, di-, tri-alkylamino group is dimethylamino styrene or dimethylaminoethyl styrene.

4. A matrix type patch for treating respiratory diseases such as asthma according to claim 1, wherein the said cationic polymer is at least one copolymers selected from a group consisting of vinyl pyrrolidone-dimethyl aminoethyl methacrylate copolymer, vinyl pyrrolidone-vinyl caprolactam-dimethyl aminoethy methacrylate copolymer and vinyl pyrrolidone methacrylamidopropyl trimethyl ammoniumchloride copolymer.

5. A matrix type patch for treating respiratory diseases such as asthma according to claim 1, wherein the content of the bronchodilators is 0.1˜10% by weight, the content of the cationic polymer is 1˜20% by weight, and the content of the said solubilizing agents is 1˜25% by weight, on the basis of the total weight of the adhesive layer containing the drug.

6. A matrix type patch for treating respiratory diseases such as asthma according to claim 1, wherein the said solubilizing agent is at least one selected from a group consisting of N-methyl-2-pyrrolidone, laurylpyrrolidone, triethanolamine, propylene glycol, glycerine, triacetine, diethyleneglycol monoethylether, fatty acid and fatty alcohol derivatives.

7. A matrix type patch for treating respiratory diseases such as asthma according to claim 2, wherein the said cationic polymer is at least one copolymers selected from a group consisting of vinyl pyrrolidone-dimethyl aminoethyl methacrylate copolymer, vinyl pyrrolidone-vinyl caprolactam-dimethyl aminoethy methacrylate copolymer and vinyl pyrrolidone methacrylamidopropyl trimethyl ammoniumchloride copolymer.
